(19) United States
(12) Patent Application Publication
(10) Pub. No.: US 2014/0228623 A1
(43) Pub. Date: Aug. 14, 2014

(54) FIXING DEVICE

(71) Applicant: TERUMO KABUSHIKI KAISHA, Tokyo (JP)

(72) Inventors: Shigeki ARIURA, Kanagawa (JP); Masakatsu Kawaura, Kanagawa (JP)

(73) Assignee: TERUMO KABUSHIKI KAISHA, Tokyo (JP)

(21) Appl. No.: 14/256,369

(22) Filed: Apr. 18, 2014

Related U.S. Application Data
(63) Continuation of application No. PCT/JP2012/073487, filed on Sep. 13, 2012.

(30) Foreign Application Priority Data
Oct. 20, 2011 (JP) 2011-231112

(51) Int. Cl. A61F 2/00

(52) U.S. Cl. CPC A61F 2/005; A61B 17/0482
USPC 600/30

(57) ABSTRACT

A fixing device includes a vaginal insertion portion of a longitudinal shape which is inserted into the vagina, a urethral-insertion member having a urethral-insertion portion of a longitudinal shape which is inserted into the urethra, and an interlock portion which is interlock means configured to interlock the vaginal insertion portion and the urethral-insertion member with each other. The vaginal insertion portion has a form of a plate and is curved. Further, the vaginal insertion portion has, as a guide portion which supports a medical tool of a longitudinal shape for movement along a longitudinal direction of the medical tool so as to guide the medical tool in the proximity of the urethra and in a direction in which the medical tool bypasses the urethra, two guide holes and which extend through the vaginal insertion portion in a thicknesswise direction of the vaginal insertion portion.

### CROSS REFERENCES TO RELATED APPLICATIONS

This application is a continuation of International Application No. PCT/JP2012/073487 filed on Sep. 13, 2012, and claims priority to Japanese Application No. 2011-231112 filed on Oct. 20, 2011, the entire content of both of which is incorporated herein by reference.

### TECHNICAL FIELD

The present invention generally relates to a fixing device.

### BACKGROUND DISCUSSION

If a person suffers from a urinary incontinence, especially from stress urinary incontinence, then urine leakage is caused by the fact that abdominal pressure is applied during a normal exercise or is applied by laughing, coughing, sneezing and the like. One of reasons for this is that the pelvic floor muscle which is a muscle for supporting the urethra is loosened, for example, as a result of childbearing or the like.

An effective treatment for urinary incontinence is a surgical treatment in which there is used, for example, a tape-shaped implant called a “sling.” The sling is indwelled inside the body and the urethra is supported by the sling. An example is disclosed in Japanese Application Publication No. 2010-99499. In order to indwell the sling inside the body, an operator would incise the vagina with a surgical knife, dissect a region between the urethra and the vagina, and communicate that dissected region and the outside with each other through an obturator foramen using a puncture needle or the like. Then, in such a state, the sling is indwelled into the dissected region in the body.

However, the method of indwelling a sling using a conventional medical tool such as a surgical knife is disadvantageous in that the procedure is rather invasive and the burden on a patient is heavy. Further, when a region between the urethra and the vagina is dissected, there is the possibility that the direction of the dissection may be wrong, resulting in failure in indwelling of the sling at an appropriate position. Further, there is the possibility that, during a procedure by an operator, the urethra, the bladder or the like may be damaged, and there is the possibility that also the operator may damage his/her fingertip.

### SUMMARY

According to one aspect, a fixing device comprises a vaginal insertion portion configured to be inserted into a vagina, the vaginal insertion portion possessing an elongated shape, and a urethral-insertion portion configured to be inserted into a urethra. The urethral-insertion portion possesses an elongated shape and is interlocked with the vaginal insertion portion. The vaginal insertion portion includes a guide portion configured to support a medical tool which possesses a longitudinal shape for movement along a longitudinal direction of the medical tool so as to guide the medical tool adjacent a urethra and along a path of movement in which the medical tool bypasses the urethra.

The fixing device can guide the elongated medical tool to a target region past the urethra in proximity of the urethra of a patient, and is quite high in safety of the patient and also in safety of an operator.

The guide portion is preferably configured to guide a plurality of the medical tools in directions, or along paths of movement, different from each other.

The guide portion preferably has a guide hole extending through the vaginal insertion portion.

Preferably, an extension line of the guide hole does not cross the urethral-insertion portion.

The guide portion of the fixing device preferably has two guide holes extending through the vaginal insertion portion, and extension lines of the two guide holes do not individually cross the urethral-insertion portion.

The extension lines of the two guide holes preferably cross each other between the vaginal insertion portion and the urethral-insertion portion.

The fixing device preferably includes adjustment means for adjusting the separation distance between the vaginal insertion portion and the urethral-insertion portion.

The urethral-insertion portion is preferably provided for movement along a longitudinal direction of the vaginal insertion portion with respect to the vaginal insertion portion.

The vaginal insertion portion of the fixing device preferably is plate-shaped.

The vaginal insertion portion is preferably curved.

The urethral-insertion portion is preferably curved in a direction same as a direction of the vaginal insertion portion.

According to another aspect, a fixing device includes a urethral-insertion portion configured to be inserted into a urethra, the urethral-insertion portion possessing an elongated shape, a restriction unit on the urethral-insertion portion that restricts a position of the urethral-insertion portion, in a longitudinal direction of the urethral-insertion portion, in the urethra, and a guide portion interlocked with the urethral-insertion portion to movably support a medical tool possessing an elongated shape for movement along a longitudinal extent of the medical tool to guide the medical tool adjacent a urethra and along a path of movement in which the medical tool bypasses the urethra.

The restriction unit is preferably disposed at the distal end side of the urethral-insertion portion with respect to a position of the urethral-insertion portion in a longitudinal direction of the urethral-insertion portion on an extension line of the guide portion.

The restriction unit is preferably an expandable and contractible balloon.

The fixing device further includes adjustment means for adjusting the separation distance between the restriction unit and a position of the urethral-insertion portion in a longitudinal direction of the urethral-insertion portion on an extension line of the guide portion.

The fixing device is preferably provided with graduations identifying the separation distance between the restriction unit and the position of the urethral-insertion portion in a longitudinal direction of the urethral-insertion portion on an extension line of the guide portion.

With the fixing device disclosed here, when an implant is buried, the burden on the patient is light and the safety of the patient is high, and also the safety of the operator is high.

In particular, by inserting the vaginal insertion portion into the vagina and inserting the urethral-insertion portion into the urethra, the position of the urethra with respect to the vaginal insertion portion is fixed to a particular position, and a medical tool of a longitudinal shape like a needle is guided by the guide portion provided on the vaginal insertion portion. Therefore, the needle can be prevented from puncturing the urethra or the bladder and can be prevented from puncturing a fingertip of the operator.

According to another aspect, a method of inserting an elongated medical tool into a living body that includes a urethra comprises inserting an elongated urethral insertion portion into the urethra of the living body, puncturing the living body with the medical tool by moving the elongated medical tool in a direction along a longitudinal extent of the medical tool, and guiding the elongated medical tool along a guide portion while the medical tool is moved in the direction along the longitudinal extent of the medical tool so that the medical tool is moved adjacent the urethra in the living body and along a path of movement in which the medical tool bypasses the urethra.

### BRIEF DESCRIPTION OF THE DRAWINGS

FIGS. 1(a)-1(e) depict a first embodiment of a fixing device disclosed here.

FIGS. 2(a)-2(e) illustrate an operation procedure of the fixing device depicted in FIGS. 1(a)-1(e).

FIGS. 3(a)-3(d) illustrate the operation procedure of the fixing device depicted in FIGS. 1(a)-1(e).

FIGS. 4(a)-4(e) is a view illustrating the operation procedure of the fixing device depicted in FIGS. 1(a)-1(e).

FIGS. 5(a)-5(c) are side elevational views depicting a needle used in a different operation procedure of the fixing device depicted in FIGS. 1(a)-1(e).

FIGS. 6(a)-6(c) illustrate the different operation procedure of the fixing device depicted in FIGS. 1(a)-1(e).

FIGS. 7(a)-7(c) illustrate the different operation procedure of the fixing device depicted in FIGS. 1(a)-1(e).

FIGS. 8(a)-8(c) illustrate the different operation procedure of the fixing device depicted in FIGS. 1(a)-1(e).

FIGS. 9(a) and 9(b) illustrate the different operation procedure of the fixing device depicted in FIGS. 1(a)-1(e).

FIGS. 10(a)-10(c) illustrate the different operation procedure of the fixing device depicted in FIGS. 1(a)-1(e).

FIGS. 11(a)-11(c) illustrate the different operation procedure of the fixing device depicted in FIGS. 1(a)-1(e).

FIGS. 12(a)-12(c) depict a second embodiment of the fixing device disclosed here.

FIGS. 13(a) and (b) depict a third embodiment of the fixing device disclosed here.

### DETAILED DESCRIPTION

Set forth below, with reference to the accompanying drawings, is a detailed description of embodiments of a fixing device representing examples of the fixing device disclosed here.

### First Embodiment

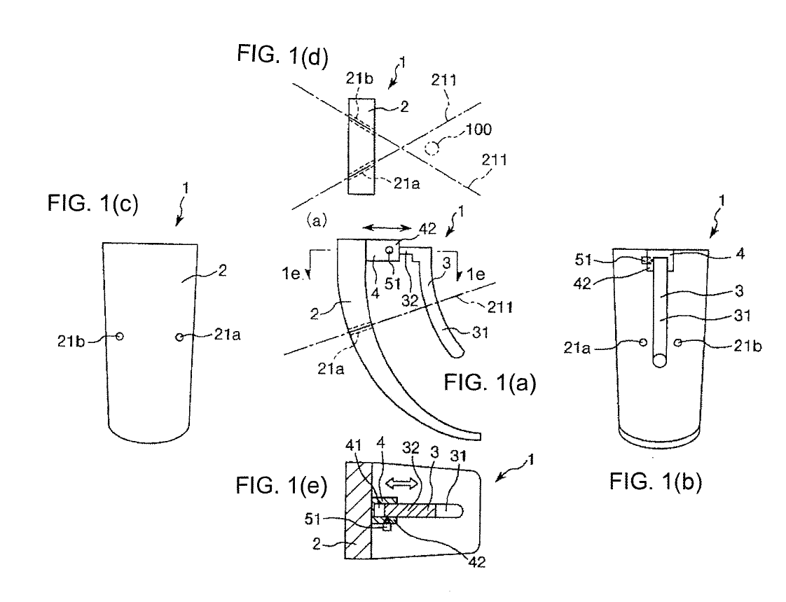

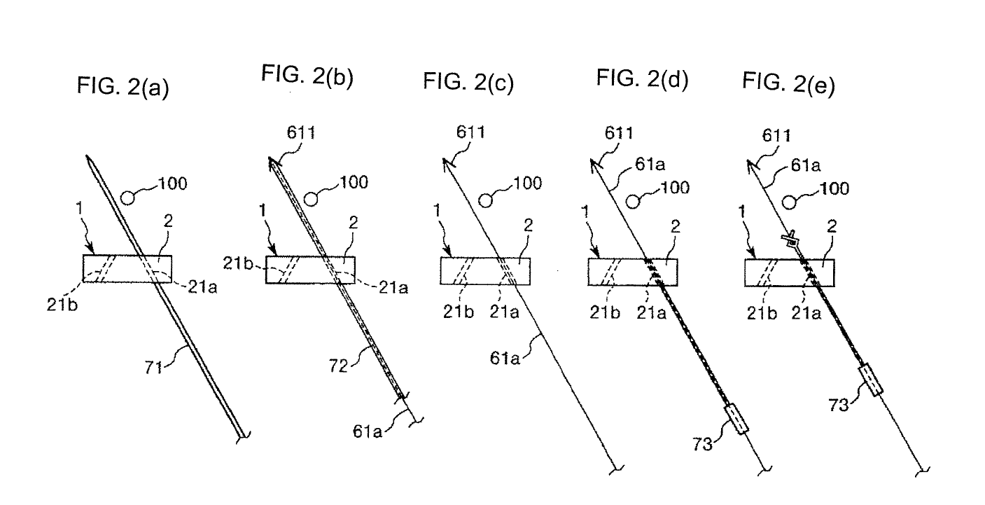

A first embodiment of the disclosed fixing device is illustrated in the various drawing figures constituting FIG. 1, where FIG. 1(a) is a side elevational view of the fixing device, FIG. 1(b) is a front elevational view of the fixing device; FIG. 1(c) is a rear elevational view of the fixing device; FIG. 1(d) is a plan view depicting a vaginal insertion portion; and FIG. 1(e) is a cross-sectional view of the fixing device taken along the section line 1e-1e in FIG. 1(a); and the various drawing figures constituting FIGS. 2 to 4 individually illustrate an operation procedure of the fixing device depicted in FIG. 1.

In the description below, the lower side and the upper side of a vaginal insertion portion and a urethral-insertion portion along their longitudinal direction in FIG. 1(a) are represented by or referred to as the “distal end” and “proximal end,” respectively, and the right upper side or the left upper side in FIGS. 2 to 4 is represented by or referred to as the “distal end” and the left lower side or the right lower side is represented by or referred to as the “proximal end.” Further, in FIG. 1(d), the urethra of a patient when it is assumed that the fixing device is mounted on the patient is indicated by a two-dot chain line. Meanwhile, in FIGS. 2 to 4, only a vaginal insertion portion of the fixing device is depicted while depiction of the other part of the fixing device is omitted.

The fixing device 1 depicted in these figures is a device used for medical treatment of urinary incontinence in a woman, namely, when an implant (living body indwelling device) is buried in the living body.

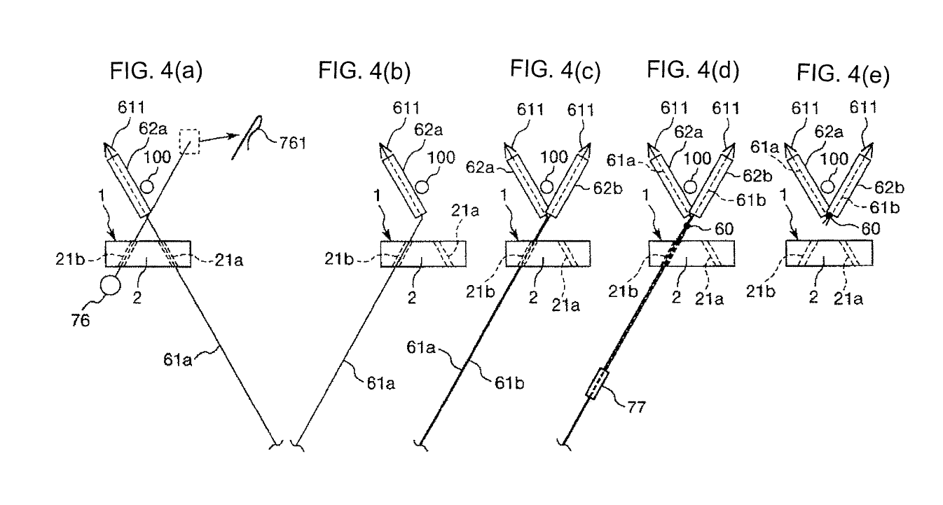

The implant is a device which can be buried for medical treatment of urinary incontinence in a woman, namely, a device which supports the urethra so as not to move to the vaginal wall side. As this implant, for example, a long (elongated) object having flexibility can be used. In the present embodiment, the implant is configured from two strings 61a and 61b and two bands 62a and 62b (refer to FIG. 4). Note that the implant is called a “sling.”

Each of the strings 61a and 61b has, at one end portion of the string, a fixation portion 611 for being fixed to the living body (biological tissue).

The fixation portions 611 are regions which, during use, puncture the living body and are individually configured such that the resistance of the fixation portions 611 when it is tried to pull them out from the living body is higher than that of any other portion of the strings 61a and 61b. In the present embodiment, each fixation portion 611 has a V shape. That is, each fixation portion 611 is a V-shaped fixation portion. The direction of “V” of the fixation portions 611 is set such that, when the strings 61a and 61b puncture the living body, the open side of the V is directed in a direction opposite to a direction in which the strings 61a and 61b advance (refer to FIGS. 2 and 4).

Further, the dimension of each of the strings 61a and 61b is not specifically limited but is set suitably. By way of example, where the cross-sectional shape of each string 61a and 61b is a circular shape, the diameter preferably is approximately 0.3 to 3 mm, and more preferably is approximately 0.5 to 2 mm.

The construction materials of the strings 61a and 61b are not especially limited, but various resin materials, fibers and so forth having biocompatibility such as, for example, polypropylene can be used.

The dimensions of the bands 62a and 62b are also not particularly limited, but are set suitably. By way of example, the width of the bands is preferably approximately 2 to 10 mm and the thickness of the bands is preferably approximately 0.3 to 1.5 mm.

Further, although the construction materials of the bands 62a and 62b are not especially limited, various resin materials, fiber knitted in meshes and so forth having biocompatibility such as, for example, polypropylene, can be used.

As depicted in FIG. 1, the fixing device 1 includes a vaginal insertion portion 2 of an elongated shape for being inserted into the vagina, a urethral-insertion member 3 having a urethral-insertion portion 31 of an elongated shape for being inserted into the urethra, and an interlock portion 4 serving as interlock means for interlocking the vaginal insertion portion 2 and the urethral-insertion member 3 with each other.

Although the shape of the vaginal insertion portion 2 is not especially limited so long as it possesses an elongated shape, the vaginal insertion portion 2 in the present embodiment has the form of a plate. That is, the vaginal insertion portion 2 is plate-shaped. Further, the width of the vaginal insertion portion 2 gradually decreases toward the distal end of the vaginal insertion portion 2, and the thickness of the vaginal insertion portion 2 gradually decreases toward the distal end of the vaginal insertion portion 2. Further, the distal portion of the vaginal insertion portion 2 is rounded. Consequently, it is possible to improve the safety to the patient.

The vaginal insertion portion 2 is also curved. Consequently, when the vaginal insertion portion 2 is inserted into the vagina, the surgical field can be directed to the front of the operator and can be widened.

Further, the vaginal insertion portion 2 has, as a guide portion for supporting a medical tool possessing an elongated shape for movement along a longitudinal direction or longitudinal extent of the medical tool, two spaced apart (separate) guide holes 21a and 21b (through holes) extending completely through the vaginal insertion portion 2 in its thicknesswise direction. The medical tool possessing the elongated shape is inserted into and used in each of the guide holes 21a and 21b. In the following description, the medical tool possessing the elongated shape is also referred to as the “medical tool.”

Each of the guide holes 21a and 21b individually supports a medical tool for movement along a longitudinal direction of the medical tool such that it guides the medical tool in the neighborhood of (adjacent to) the urethra and in a direction (along a path of movement) in which the medical tool bypasses the urethra. In this case, the guide holes 21a and 21b are configured so as to guide two medical tools in different directions (along different paths) from each other.

Further, although the shape of each of the guide holes 21a and 21b is not especially limited, in the present embodiment, it is assumed that the medical tool has a linear shape, and therefore, each of the guide holes 21a and 21b has a linear shape. If it is assumed that a medical tool has a curved shape, then each of the guide holes 21a and 21b preferably has a curved shape.

As the medical tool possessing an elongated shape, for example, a needle 71 hereinafter described, other needles, sheaths, catheters, forceps, endoscopes, electrodes configured to apply radio waves to the tissue to heat the tissue, optical fibers configured to irradiate laser light or the like upon the tissue, probes for applying ultrasonic waves to the tissue and so forth are available. In the following description, a case in which the medical tool is the needle 71 is described as a representative example.

Further, an extension line 211 of each of the guide holes 21a and 21b (central axis of the guide holes 21a, 21b) does not cross or intersect the urethral-insertion portion 31. In other words, the extension line 211 is displaced from the urethral-insertion portion 31. Consequently, if the needle 71 inserted in each of the guide holes 21a and 21b is moved in a direction toward the distal end, then the needle 71 does not contact the urethra and can be prevented from damaging the urethra.

Further, the extension lines 211 of the guide holes 21a and 21b cross or intersect each other at a position or location between the vaginal insertion portion 2 and the urethral-insertion portion 31 as shown in FIG. 1(d).

The urethral-insertion member 3 has the urethral-insertion portion 31, and a protruding portion 32 formed at the proximal portion of the urethral-insertion portion 31 and projecting from the urethral-insertion portion 31 toward the left side in FIG. 1(a).

The shape of the urethral-insertion portion 31 is not limited so long as it is an elongated shape. In the illustrated, embodiment, the urethral-insertion portion 31 has a form of a bar (i.e., bar-shaped). Further, the distal portion of the urethral-insertion portion 31 is rounded. Consequently, the safety of the patient can be improved.

Further, the urethral-insertion portion 31 is curved in the same direction as that of the vaginal insertion portion 2. The curvature of the urethral-insertion portion 31 is set equal to that of the vaginal insertion portion 2. Further, the posture of the urethral-insertion portion 31 is set such that the separation distance between the urethral-insertion portion 31 and the vaginal insertion portion 2 is fixed along the longitudinal direction of the urethral-insertion portion 31.

The interlock portion 4 is fixed to the right side of the proximal portion of the vaginal insertion portion 2 in FIG. 1(a).

A bottomed hole 41 is formed along the leftward and rightward direction in FIG. 1(a) in the interlock portion 4. The hole 41 is open at the right side in FIG. 1(a).

In the hole 41, the protruding portion 32 of the urethral-insertion member 3 is inserted for sliding movement in the longitudinal direction of the protruding portion 32. As the urethral-insertion member 3 moves in the longitudinal direction of the protruding portion 32 with respect to the interlock portion 4, the separation distance between the vaginal insertion portion 2 and the urethral-insertion portion 31 is changed. Accordingly, adjustment means for adjusting the separation distance between the vaginal insertion portion 2 and the urethral-insertion portion 31 is configured from the hole 41 of the interlock portion 4 and the protruding portion 32 of the urethral-insertion member 3. That is, the hole 41 of the interlock portion 4 and the protruding portion 32 of the urethral-insertion member 3 represent an example of an adjustment means for adjusting the separation distance between the vaginal insertion portion 2 and the urethral-insertion portion 31. Since patients have individual differences and the separation distance between the vagina and the urethra sometimes differs depending upon the patient, the separation distance between the vaginal insertion portion 2 and the urethral-insertion portion 31 can be suitably adjusted by the adjustment means so as to match the patient.

Whatever is set as the separation distance between the vaginal insertion portion 2 and the urethral-insertion portion 31, in a case that the extension lines 211 of the guide holes 21a and 21b are displaced from the urethral-insertion portion 31, and the vaginal insertion portion 2 and the urethral-insertion portion 31 are positioned nearest to each other, the extension lines 211 cross each other between the vaginal insertion portion 2 and the urethral-insertion portion 31.

Further, the fixing device 1 has a male screw 51, and a female screw portion 42 having a female screw for meshing with the male screw 51 is formed in a region of the interlock portion 4 corresponding to the hole 41 at a right side end portion of the interlock portion 4 in FIG. 1(a).

If the male screw 51 is rotated in a predetermined direction, the distal end of the male screw 51 contacts, under pressure, the protruding portion 32 of the urethral-insertion member 3 so that movement of the urethral-insertion member 3 with respect to the interlock portion 4 is blocked. On the other hand, if the male screw 51 is rotated in a direction opposite to the direction, then the distal end of the male screw 51 is separated from the protruding portion 32 so that movement of the urethral-insertion member 3 with respect to the interlock portion 4 is permitted.

Note that a locking unit for performing changeover between a state in which the urethral-insertion member 3 can move with respect to the interlock portion 4 and another state in which the movement of the urethral-insertion member 3 is blocked is configured from the male screw 51 and the female screw portion 42.

Now, an operation procedure of the fixing device 1, namely, a procedure when the implant configured from the two strings 61a and 61b and the two bands 62a and 62b is buried into the living body, is described.

First, the fixing device 1 is mounted on a patient. In particular, the vaginal insertion portion 2 of the fixing device 1 is inserted into the vagina, and the urethral-insertion portion 31 is inserted into the urethra. Consequently, the position of the urethra with respect to the vaginal insertion portion 2 is fixed at a particular position. The position of the urethral-insertion portion 31 with respect to the interlock portion 4 is then changed as occasion demands to adjust the separation distance between the vaginal insertion portion 2 and the urethral-insertion portion 31. In particular, as occasion demands, the male screw 51 is rotated in a loosening direction to move the urethral-insertion member 3 to the left side or the right side in FIG. 1(a) with respect to the interlock portion 4, and the male screw 51 is rotated in a tightening direction to establish a state in which movement of the urethral-insertion member 3 with respect to the interlock portion 4 is blocked or prevented. At this time, a portion of the vaginal insertion portion 2 including at least the guide holes 21a and 21b is preferably held in close contact with the vaginal wall at the urethra side.

Then, the needle 71 is used to form two puncture holes in the patient. As this needle 71, a linear needle is used in the present embodiment. The needle 71 may be solid or may be in the form of a pipe.

Further, one of the two puncture holes is a bottomed hole extending in the proximity of the urethra 100 from the vaginal wall past the left side of the urethra 100 in FIG. 2(a) toward an obturator foramen not depicted at the left side in FIG. 2(a). The other of the two puncture holes is a bottomed hole which extends in the proximity of the urethra 100 from within the vagina past the right side of the urethra 100 in FIG. 2(a) toward an obturator foramen not depicted at the right side in FIG. 2(a). The puncture holes cross each other and are each formed to some position between a position a little nearer from the obturator foramen and another position a little farther than the obturator foramen.

The procedures for forming the two puncture holes are similar to each other and either one of them may be carried out first. In the following description, a case in which the puncture hole which extends from the vaginal wall in the proximity of the urethra 100 past the left side of the urethra 100 in FIG. 2(a) toward the obturator foramen not shown at the left side in FIG. 2(a) is formed first is described as a representative.

Then, the needle 71 is inserted into the guide hole 21a of the vaginal insertion portion 2 at the right side in FIG. 2(a), and the needle 71 is moved forward in a direction toward the distal end, inserted into the vagina and further moved by a predetermined distance in a direction toward the distal end. Consequently, the needle 71 is guided by the guide hole 21a, punctures the vaginal wall from within the vagina, bypasses the urethra 100, namely, passes the left side of the urethra 100 in FIG. 2(a) in the proximity of the urethra 100 and moves to the predetermined position. In this manner, by virtue of the guide hole 21a, the needle 71 can be safely prevented from puncturing the urethra 100.

Then, the needle 71 is pulled out. In this manner, the puncture hole passing the left side of the urethra 100 in FIG. 2(a) in the proximity of the urethra 100 from the vaginal wall is formed in the patient.

Then, as depicted in FIG. 2(b), the string 61a is accommodated in a tubular needle 72, and the fixation portion 611 of the string projects from the distal end of the needle 72. Then, the needle 72 is inserted into the guide hole 21a of the vaginal insertion portion 2, and the needle 72 is moved in a direction toward the distal end, inserted into the vagina and further moved by a predetermined distance in a direction toward the distal end. Consequently, the needle 72 is guided by the guide hole 21a, inserted into the puncture hole and moved in the puncture hole to the bottom of the puncture hole in the direction toward the distal end.

Then, the needle 72 is pulled out as depicted in FIG. 2(c). Thereupon, the fixation portion 611 of the string 61a is caught by the living body so that the fixation portion 611 can be prevented from being pulled out from the living body. The fixation portion 611 of the string 61a is fixed to the living body in this manner.

Thereafter, as depicted in FIGS. 2(d) and 2(e), the tubular elongated portion of a dissection device 73, which has the tubular elongated portion and scissors provided at the distal portion of the elongated portion, is threaded over the string 61a. Then, in a state in which the scissors are closed, the dissection device 73 is inserted into the guide hole 21a of the vaginal insertion portion 2, moved in a direction toward the distal end along the string 61a, inserted into the vagina and further moved by a predetermined distance in the direction toward the distal end. Consequently, the dissection device 73 is guided by the guide hole 21a and the string 61a, inserted into the puncture hole and moved to a place, at which dissection is to be carried out, in the direction toward the distal end in the puncture hole.

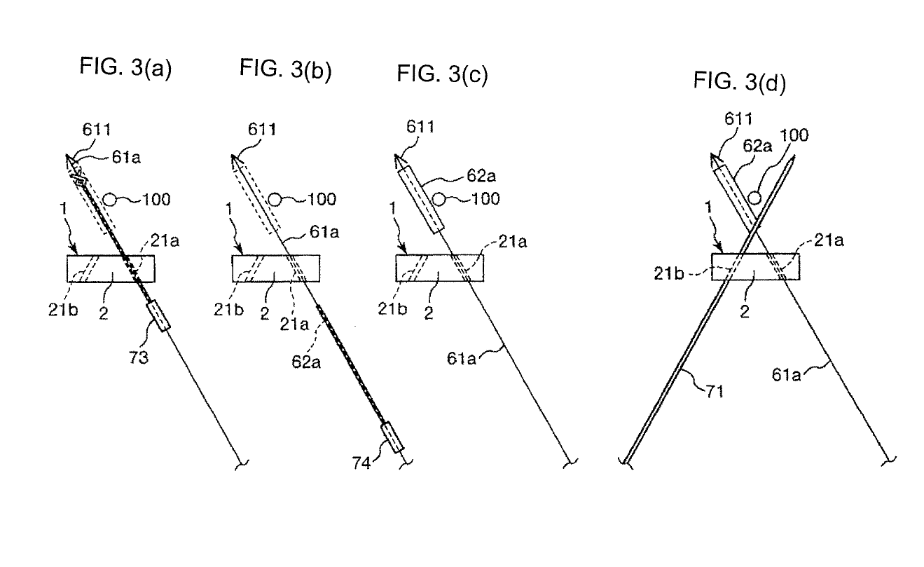

Then, as depicted in FIG. 3(a), the biological tissue between the urethra 100 and the vaginal wall or the like is dissected by the dissection device 73 to form a space into which the band 62a is to be inserted. In FIGS. 3(a) and 3(b), the dissection region to be dissected by the dissection device 73 is indicated by broken lines.

Then, the dissection device 73 is pulled out.

Next, as depicted in FIG. 3(b), the band 62a is accommodated into or at the distal portion of a sheath 74, and the sheath 74 is threaded over the string 61a. Then, the sheath 74 is inserted into the guide hole 21a of the vaginal insertion portion 2, and this sheath 74 is moved in a direction toward the distal end along the string 61a, inserted into the vagina and further moved by a predetermined distance in the direction toward the distal end. Consequently, the sheath 74 is guided by the guide hole 21a and the string 61a, inserted into the puncture hole and moved to the place dissected by the dissection device 73 in the direction toward the distal end in the puncture hole.

Then, a pusher which can push out the band 62a is inserted into the sheath 74, and while the sheath 74 is moved in a direction toward the proximal end, the band 62a in the sheath 74 is pushed out from the distal end of the sheath 74 by the pusher. Then, the sheath 74 and the pusher are pulled out. In this manner, the band 62a is inserted into (positioned in) the place dissected by the dissection device 73 as depicted in FIG. 3(c).

Next, as depicted in FIG. 3(d), the needle 71 is inserted into the guide hole 21b of the vaginal insertion portion 2 at the left side in FIG. 3(d), and the needle 71 is moved in a direction toward the distal end, inserted into the vagina and further moved by a predetermined distance in the direction toward the distal end. Consequently, the needle 71 is guided by the guide hole 21b, punctures the vaginal wall from within the vagina, bypasses the urethra 100, namely, passes to the right side of the urethra 100 in FIG. 3(d) in the neighborhood of (adjacent to) the urethra 100, and moves to a predetermined position. In this manner, the needle 71 can be safely prevented from puncturing the urethra 100 by the guide hole 21b.

The needle 71 is then pulled out. In this manner, the puncture hole which passes the right side of the urethra 100 in FIG. 3(d) in the proximity of the urethra 100 from the vaginal wall is formed in the patient.

Next, as depicted in FIG. 4(a), a string catching device 76 having a hook 761 at the distal portion of the string catching device 76 is inserted into the guide hole 21b of the vaginal insertion portion 2, and the string catching device 76 is moved in a direction toward the distal end, inserted into the vagina and further moved by a predetermined distance in the direction toward the distal end. Consequently, the string catching device 76 is guided by the guide hole 21b and inserted into the puncture hole and then moved to a position farther than the string 61a in the direction toward the distal end in the puncture hole.

Then, as depicted in FIG. 4(b), the string catching device 76 is moved in the proximal direction toward the proximal end, and the string 61a is caught by the hook 761 to pull out the string 61a. Consequently, the string 61a is pulled out from the puncture hole which passes the left side of the urethra 100 in FIG. 4(b) and from the guide hole 21a, and is inserted into the puncture hole which passes the right side of the urethra 100 in FIG. 4(b) and into the guide hole 21b so that the string 61a is guided to the outside.

In the following description, since the procedure to halfway is similar to the procedure described above, the description is given with the illustration omitted.

Then, the other string 61b is accommodated in the needle 72, and the fixation portion 611 projects from the distal end of the needle 72. The needle 72 is then inserted into the guide hole 21b of the vaginal insertion portion 2, and the needle 72 is moved in a direction toward the distal end, inserted into the vagina and further moved by a predetermined distance in the direction toward the distal end. Consequently, the needle 72 is guided by the guide hole 21b, inserted into the puncture hole and moved in the puncture hole to the bottom of the puncture hole in the direction toward the distal end.

Then, the needle 72 is pulled out. Thereupon, the fixation portion 611 of the string 61b is caught by the living body so that the fixation portion 611 can be prevented from being pulled out from the living body. The fixation portion 611 of the string 61b is fixed to the living body in this manner.

The tubular elongated portion of a dissection device 73 is then threaded over the string 61b. Next, in a state in which the scissors are closed, the dissection device 73 is inserted into the guide hole 21b of the vaginal insertion portion 2, and this dissection device 73 is moved in a direction toward the distal end along the string 61b, inserted into the vagina and further moved by a predetermined distance in the direction toward the distal end. Consequently, the dissection device 73 is guided by the guide hole 21b and the string 61b, inserted into the puncture hole and moved to a place, at which dissection is to be carried out, in the direction toward the distal end in the puncture hole.

Then, the biological tissue between the urethra 100 and the vaginal wall or the like is dissected by the dissection device 73, and a space into which the other band 62b is to be inserted is formed.

The dissection device 73 is then pulled out.

Next, the other band 62b is accommodated into the distal portion in the sheath 74, and the sheath 74 is threaded over the string 61b. Then, the sheath 74 is inserted into the guide hole 21b of the vaginal insertion portion 2, and this sheath 74 is moved in a direction toward the distal end along the string 61b, inserted into the vagina and further moved by a predetermined distance in the direction toward the distal end. Consequently, the sheath 74 is guided by the guide hole 21b and the string 61b, inserted into the puncture hole, and moved to the place dissected by the dissection device 73 in the direction toward the distal end in the puncture hole.

Then, a pusher is inserted into the sheath 74, and while the sheath 74 is moved in the direction toward the proximal end, the band 62b in the sheath 74 is pushed out from the distal end of the sheath 74 by the pusher. Then, the sheath 74 and the pusher are pulled out. In this manner, the band 62b is inserted into the place dissected by the dissection device 73 as depicted in FIG. 4(c).

Then, an apparatus is used to put the two strings 61a and 61b together to form a knot 60 (refer to FIG. 4(d)). As a knotting method for the knot 60, a knotting method by which a knot can be moved only in one direction such as, for example, clinch knot is applied. Then, the knot 60 is moved in the direction toward the distal end. Thereupon, the operator would adjust the tightening degree of the strings 61a and 61b by the movement of the knot 60. The knot 60 and the bands 62a and 62b pushed in the direction toward the distal end by the knot 60 are attached to the biological tissue around the urethra 100, and by the tensile force of the strings 61a and 61b, the urethra 100 is pulled in a direction in which it is separated from the vaginal wall and the urethra 100 is supported by the strings 61a and 61b and the bands 62a and 62b.

Then, as depicted in FIG. 4(d), the strings 61a and 61b are threaded into a tubular elongated portion of a string cutting tool 77 which has the tubular elongated portion and a string cutting unit provided at the distal end of the elongated portion. Then, the string cutting tool 77 is inserted into the guide hole 21b of the vaginal insertion portion 2, and the string cutting tool 77 is moved in a direction toward the distal end along the strings 61a and 61b, inserted into the vagina and further moved by a predetermined distance in the direction toward the distal end. Consequently, the string cutting tool 77 is guided by the guide hole 21b and the strings 61a and 61b and inserted into the puncture hole and is moved in the direction toward the distal end to a position before the knot 60 in the puncture hole.

Following this, as depicted in FIG. 4(e), a region of the strings 61a and 61b at the proximal end side with respect to the knot 60 is cut by the string cutting tool 77, and unnecessary portions of the strings 61a and 61b are cut away. The string cutting tool 77 is then pulled out, and predetermined suturing or the like is carried out, thereby ending or completing the procedure.

As described above, with the present fixing device 1, indwelling an implant can be achieved by a procedure that is minimally invasive such as puncture by the needle 72, and an incision or the like that is highly invasive need not be carried out. Therefore, the burden on the patient is rather light and also the safety of the patient is high.

Further, the needle 71 can be prevented from puncturing the urethra 100. Also the operator itself can safely prevent a fingertip from being punctured by a needle or the like.

While the number of guide holes in the present embodiment is two, the number of guide holes may be one or three or more.

Further, the guide holes of the vaginal insertion portion may be open at the side so that they are groove-shaped guide holes or guides.

Further, while, in the present embodiment, the vaginal insertion portion 2 and the urethral-insertion member 3 (urethral-insertion portion 31) are interlocked indirectly with each other through the interlock portion 4, the interlock is not limited to this, but the vaginal insertion portion 2 and the urethral-insertion member 3 (urethral-insertion portion 31) may be interlocked directly with each other.

Further, while, in the present embodiment, the puncture hole formed in the patient by the needle 71 does not extend through the living body, the puncture hole is not limited to this and may be a through-hole.

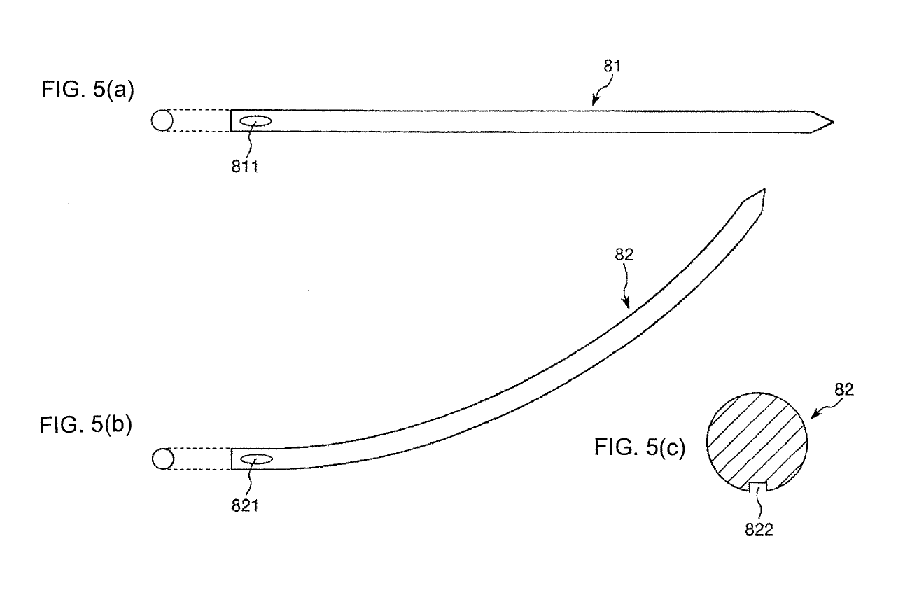

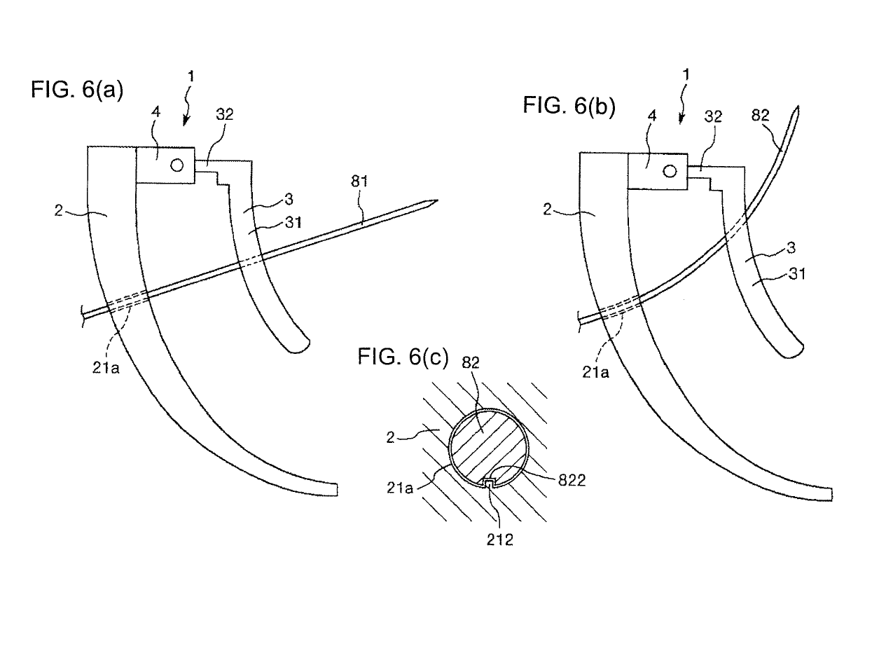

FIG. 5 is a side elevational view depicting a needle used in a different operation procedure of the fixing device depicted in FIG. 1, and FIGS. 6 to 11 are views illustrating a different operational procedure of the fixing device depicted in FIG. 1. Further, FIG. 6(a) is a side elevational view of the fixing device; FIG. 6(b) is a side elevational view of the fixing device; and FIG. 6(c) is a cross-sectional view in the proximity of the guide hole 21a of the fixing device in FIG. 6(b).

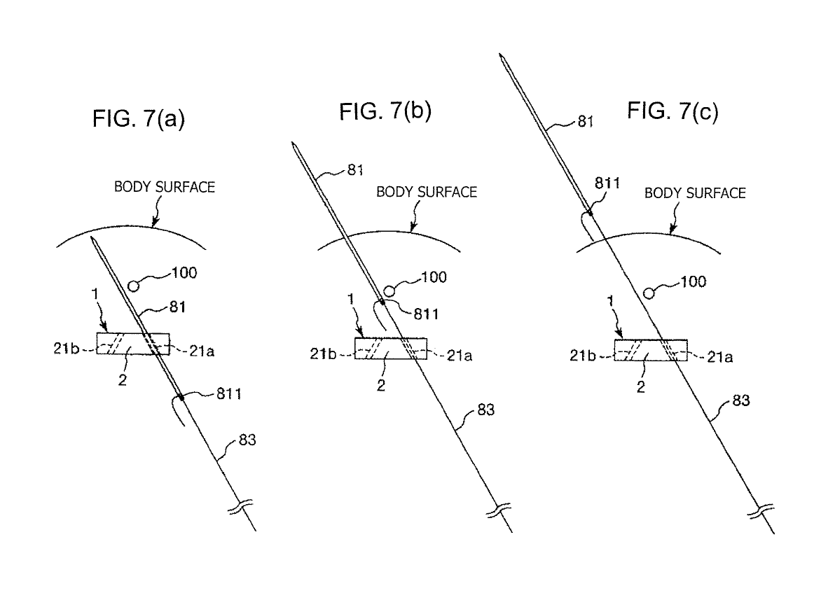

In the following description, the right upper side or the left upper side in FIGS. 7 to 11 are referred to as the “distal end,” and the left lower side or the right lower side is referred to as the “proximal end.” Further, in FIGS. 7 to 11, only the vaginal insertion portion of the fixing device is depicted while depiction of the other portion is omitted.

The following description focuses primarily on aspects of the operational procedures of the fixing device 1 which differ relative to the embodiment described above, and aspects of the operational procedures of the fixing device 1 previously described will not be repeated in detail.

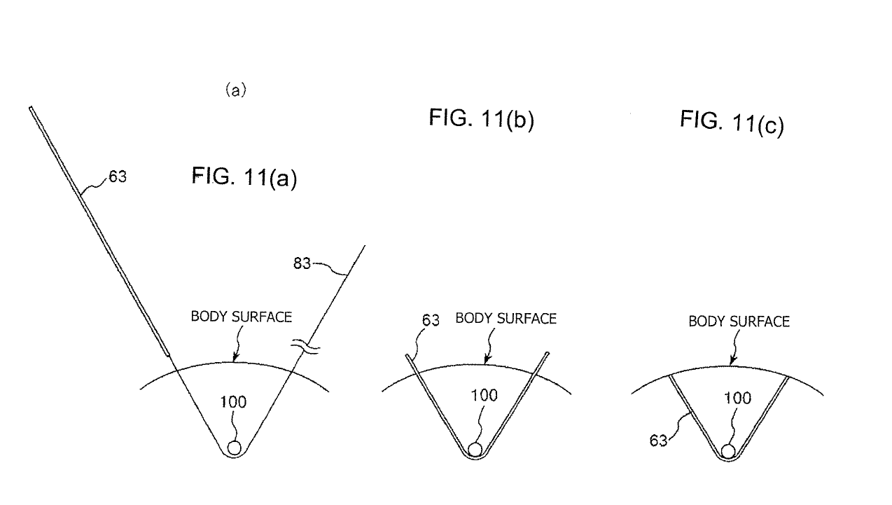

First, an implant is configured from a single band 63 as shown in FIG. 11(a).

Further, as the medical tool which possesses an elongated shape, a linear needle 81 depicted in FIG. 5(a) or a needle 82 depicted in FIG. 5(b) is used. Although the needles 81 and 82 are individually solid, they may otherwise be tubular. The needle 81 and the needle 82 are selectively used in accordance with various conditions.

Further, as depicted in FIG. 5(a), the needle 81 has a hole 811 at the proximal end of the needle 81. Further, the distal portion of the needle 81 has a conical shape.

FIG. 5(b) shows that the needle 82 has a hole 821 at the proximal end of the needle 82. As depicted in FIG. 5(c), a groove 822 is formed on the needle 82 along the longitudinal direction. The distal portion of the needle 82 has a conical shape.

Further, in the fixing device 1 which is used when the needle 82 is used, the guide hole 21a of the vaginal insertion portion 2 is curved corresponding to the needle 82 as depicted in FIGS. 6(b) and 6(c). In the guide hole 21a of the vaginal insertion portion 2, a rib 212 configured to be inserted into or positioned in the groove 822 of the needle 82 is formed. When the needle 82 is inserted into the guide hole 21a, the rib 212 is inserted into of positioned in the groove 822. Consequently, the needle 82 is restricted in its moving direction and blocked against rotation around the axis of the needle 82 by the engagement of the guide hole 21a and the groove 822.

The guide hole 21b of the vaginal insertion portion 2 is similarly curved corresponding to the needle 82 similar to the guide hole 21a, and a rib for being inserted into or positioned in the groove 822 of the needle 82 is formed in the guide hole 21b.

Now, an operation procedure of the fixing device 1, namely a procedure when the implant configured from the band 63 is buried into the living body, is described. In the following description, a case in which the needle 81 is used from between the needle 81 and the needle 82 is described.

First, the fixing device 1 is mounted on the patient in a similar manner as in the aforementioned first embodiment.

Then, as depicted in FIG. 7(a), a guide wire 83 is threaded into the hole 811 of the needle 81, and the distal end of the guide wire 83 is connected for disconnection to the proximal portion of the needle 81.

Then, the needle 81 is inserted into the guide hole 21a of the vaginal insertion portion 2 at the right side in FIG. 7(a), and the needle 81 is moved in a direction toward the distal end, inserted into the vagina and further moved in the direction toward the distal end until the needle 81 projects from the body surface of the patient. Consequently, the needle 81 is guided by the guide hole 21a, punctures the vaginal wall from within the vagina, bypasses the urethra 100, namely, passes the left side of the urethra 100 in FIG. 7(a) in the proximity of (adjacent to) the urethra 100, is inserted into the obturator foramen, punctures the biological tissue from the obturator foramen to the body surface and projects outwardly from the inguinal region or a region in the proximity of the inguinal region as depicted in FIG. 7(b).

Then, the needle 81 is pulled out from the living body as depicted in FIG. 7(c). The distal portion of the guide wire 83 is then separated from the proximal portion of the needle 81.

Although description here is given of a case in which the needle 81 is inserted into the guide hole 21a of the vaginal insertion portion 2 first, the operation or manner of use is not limited to this, as it is a matter of course that the needle 81 may be inserted into the guide hole 21b of the vaginal insertion portion 2 at the left side in FIG. 7(a) first.

Then, a space for inserting the band 63 is formed using a dissection device. The dissection device is moved to a target position along the guide wire 83.

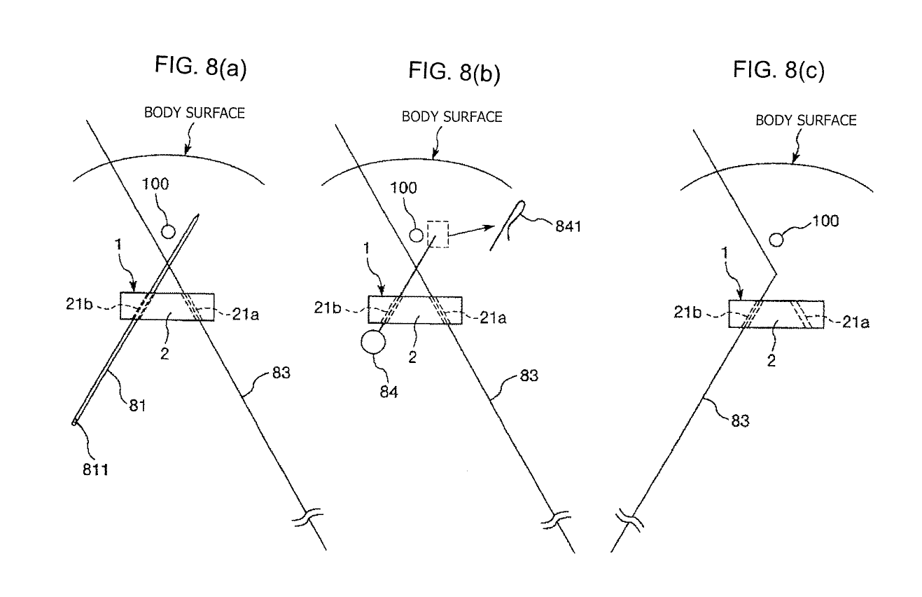

Then, as depicted in FIG. 8(a), the needle 81 is inserted into the guide hole 21b of the vaginal insertion portion 2 at the left side in FIG. 8(a), and the needle 81 is moved in a direction toward the distal end, inserted into the vagina and further moved by a predetermined distance in the direction toward the distal end. Consequently, the needle 81 is guided by the guide hole 21b, punctures the vaginal wall from within the vagina, bypasses the urethra 100, namely, passes the right side of the urethra 100 in FIG. 8(a) in the proximity of the urethra 100, and moves to the predetermined position. In this manner, the guide hole 21b can safely prevent the needle 81 from punctuating the urethra 100.

Then, the needle 81 is pulled out. In this manner, the puncture hole which passes to the right side of the urethra 100 in FIG. 8(a) in the proximity of (adjacent to) the urethra 100 from within the vagina is formed in the patient. This puncture hole crosses the previously formed puncture hole, which passes to the left side of the urethra 100 in FIG. 8(a) in the proximity of (adjacent to) the urethra 100 from within the vagina and opens to the body surface, at a position before the urethra 100.

The puncture hole formed later need not necessarily be formed so as to pass the urethra 100, but should preferably be formed to a position farther than (distally of) the guide wire 83.

Then, as depicted in FIG. 8(b), a guide wire catching device 84 is inserted into the guide hole 21b of the vaginal insertion portion 2, and this guide wire catching device 84 is moved in a direction toward the distal end, inserted into the vagina and further moved by a predetermined distance in the direction toward the distal end. Consequently, the guide wire catching device 84 is guided by the guide hole 21b, inserted into the puncture hole and moved in the direction toward the distal end to a position farther than the guide wire 83 in the puncture hole. Where the curved needle 82 is used, preferably the guide wire catching device 84 is curved with a curvature equal to that of the needle 82 or has elasticity by which it is curved to a curvature equal to that of the needle 82.

Then, as depicted in FIG. 8(c), the guide wire catching device 84 having a hook 841 at the distal portion of the guide wire catching device 84 is moved in a direction toward the proximal end, and the guide wire 83 is hooked by the hook 841, whereafter the guide wire catching device 84 is pulled out. Consequently, the guide wire 83 is pulled out from the puncture hole, which passes to the left side of the urethra 100 in FIG. 8(c), and the guide hole 21a and inserted into the puncture hole, which passes to right side of the urethra 100 in FIG. 8(c), and the guide hole 21b and then guided to the outside.

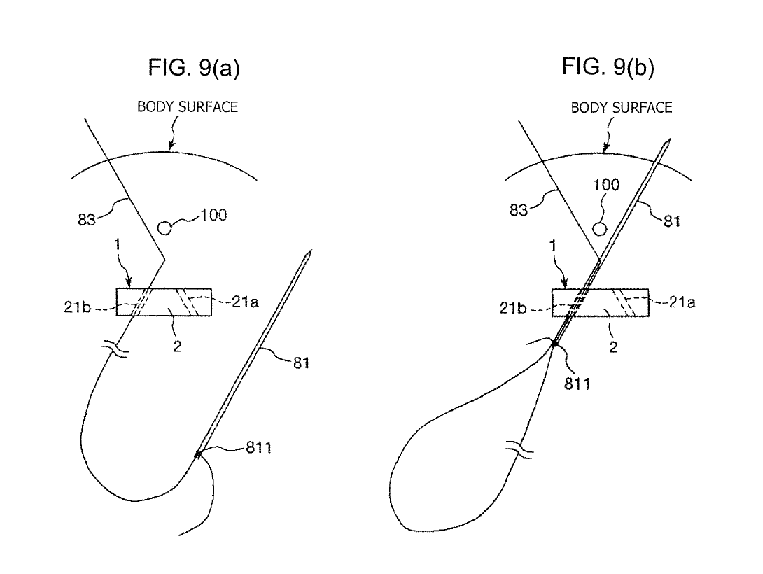

Then, as depicted in FIG. 9(a), the guide wire 83 is threaded into the hole 811 of the needle 81, and the proximal portion of the guide wire 83 is connected for disconnection to the proximal portion of the needle 81.

Then, as depicted in FIG. 9(b), the needle 81 is inserted into the guide hole 21b of the vaginal insertion portion 2 at the left side in FIG. 9(b), and the needle 81 is moved in a direction toward the distal end, inserted into the vagina and further moved in the direction toward the distal end until the needle 81 projects from (outside of) the body surface of the patient. Consequently, the needle 81 is guided by the guide hole 21b, punctures the vaginal wall from within the vagina, bypasses the urethra 100, namely, passes to the right side of the urethra 100 in FIG. 9(b) in the proximity of (adjacent to) the urethra 100, passes through the obturator foramen, punctures the biological tissue from the obturator foramen to the body surface and projects to the outside of the body from the inguinal region or a region in the proximity of the inguinal region.

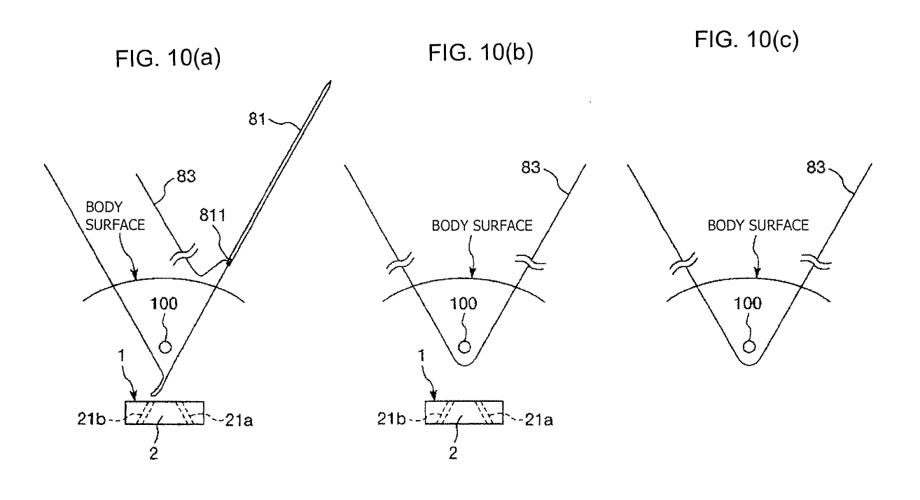

Then, the needle 81 is pulled out from the living body as depicted in FIG. 10(a). Next, as depicted in FIG. 10(b), the distal portion of the guide wire 83 is separated from the proximal portion of the needle 81. Consequently, the guide wire 83 is placed into a state in which, in a state in which the urethra 100 is caught, both end portions of the guide wire 83 project to the outside of the body.

Then, a dissection device is used to dissect the tissue around the guide wire 83 in order to form a space for inserting the band 63. The dissection device is moved to a target position along the guide wire 83.

Then, the fixing device 1 is removed from the patient as depicted in FIG. 10(c).

Next, as depicted in FIG. 11(a), the band 63 is disconnectably connected at one of the end portions of the band 63 to one of end portions of the guide wire 83, and the other end portion of the guide wire 83 is pulled. Consequently, as depicted in FIG. 11(b), the band 63 moves together with the guide wire 83, and the band 63 is placed into a state in which, in a state in which the urethra 100 is caught, the opposite end portions of the band 63 individually project to the outside of the body.

Then, the opposite end portions of the band 63 are pulled individually by predetermined force. Consequently, the urethra 100 is pulled in a direction in which it is separated from the vaginal wall by the tensile force of the band 63, and the urethra 100 is supported by the band 63.

Then, as depicted in FIG. 11(c), unnecessary portions of the band 63 are cut away, and predetermined suturing and so forth are carried out, thereby ending the procedure.

### Second Embodiment

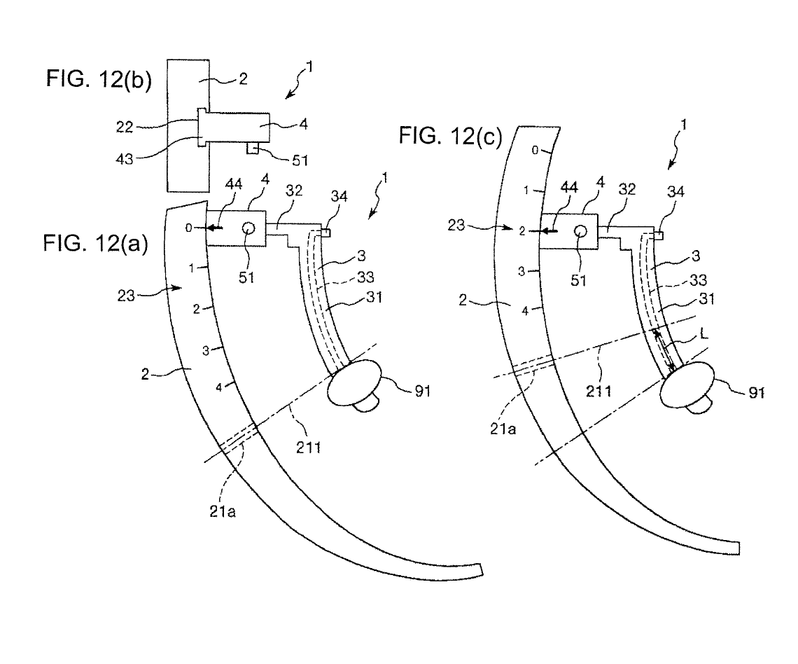

The drawing figures constituting FIG. 12 depict a second embodiment of the fixing device representing another example of the disclosed fixing device, wherein FIGS. 12(a) and 12(c) are side elevational views of the fixing device and FIG. 12(b) is a plan view of the fixing device. In FIG. 12(b), only the vaginal insertion portion, the interlock member and the male screw of the fixing device are depicted while depiction of the other region is omitted.

In the following description, the lower side and the upper side of a vaginal insertion portion and a urethral-insertion portion in FIGS. 12(a) and 12(c) along a longitudinal direction of them are referred to as the “distal end” and the “proximal end,” respectively.

The following description of the second embodiment focuses principally on the differences relative to the first embodiment described above, and features of the second embodiment that are the same as those described above in the first embodiment are identified by common reference numerals and a detailed description of such features is not repeated.

As depicted in FIGS. 12(a)-(d), the fixing device 1 of the second embodiment includes an expandable and contractible balloon 91 serving as a restriction unit, which restricts the position of the urethral-insertion portion 31 in the longitudinal direction in the urethra, at the distal portion of the urethral-insertion portion 31. When the fixing device 1 is used, the balloon 91 is inserted into the bladder of a patient. The balloon 91 is caught, in an expanded state of the balloon, by the bladder neck to fix the position of the urethral-insertion portion 31 with respect to the bladder and the urethra.

The balloon 91 is disposed at the distal end side of the urethral-insertion portion and is disposed at distal end side with respect the position of the urethral-insertion portion 31 in the longitudinal direction in the urethral-insertion portion 31 on the extension lines 211 of the guide holes 21a and 21b. That is, the balloon 91 is positioned at a location on the urethral-insertion portion 31 that is distal of the location at which an imaginary extension line of the guide holes 21a, 21b crosses or intersects the urethral-insertion portion 31. Consequently, the bladder can be prevented from being punctured by the needle 71 or the like.

Further, a lumen 33 is formed in the urethral-insertion portion 31. The distal end of the lumen 33 opens into the balloon 91 (i.e., communicates with the balloon interior) while the proximal end of the lumen 33 is open to a side face of the urethral-insertion portion 31 at the proximal portion. A port 34 which communicates with the proximal end of the lumen 33 is formed at the proximal portion of the urethral-insertion portion 31.

A balloon expanding tool like, for example, a syringe is connected to the port 34, and working fluid supplied from the balloon expanding tool is fed into or pulled out from the balloon 91 through the lumen 33 to carry out expansion or contraction of the balloon 91. Examples of the working fluid for balloon expansion include various liquids such as physiological salt solution, gas and so forth can be used.

Note that the restriction unit which restricts the position of the urethral-insertion portion 31 in the urethra is not limited to the balloon 91. For example, the restriction unit may be configured from the urethral-insertion portion 31 which is partly curved, an element which grasps part of the surface of the urethral tissue or the like.

Further, the interlock portion 4 is disposed for movement along the longitudinal direction of the vaginal insertion portion 2 with respect to the vaginal insertion portion 2. Consequently, the urethral-insertion member 3, namely, the urethral-insertion portion 31, moves integrally with the interlock portion 4 along the longitudinal direction of the vaginal insertion portion 2 with respect to the vaginal insertion portion 2.

In particular, a groove 22 which extends along the longitudinal direction of the vaginal insertion portion 2 is located on a face of the vaginal insertion portion 2 at the right side in FIG. 12(a). Further, a protruding portion 43 which is inserted in the groove 22 is located at an end portion of the interlock portion 4 at the left side in FIG. 12(a). Consequently, the moving direction of the interlock portion 4 is restricted, and the interlock portion 4 moves along the groove 22, namely, along the longitudinal direction of the vaginal insertion portion 2.

Further, when the interlock portion 4 moves along the longitudinal direction of the vaginal insertion portion 2, the separation distance L between the balloon 91 and the position of the extension lines 211 of the guide holes 21a and 21b in the urethral-insertion portion 31 in the longitudinal direction of the urethral-insertion portion 31 is changed. Accordingly, the protruding portion 43 of the interlock portion 4 and the groove 22 of the vaginal insertion portion 2 represent an example of adjustment means for adjusting the separation distance L.

Further, graduations 23 indicating the separation distance L between the balloon 91 and the location along longitudinal extent of the urethral-insertion portion 31 at which the extension lines 211 of the guide holes 21a and 21b cross or intersect the urethral-insertion portion 31 is provided on a side face of the vaginal insertion portion 2. Meanwhile, on a side face of the interlock portion 4, an arrow mark 44 for pointing to a graduation 23 is provided. Further, the graduations 23 are configured such that the graduation 23 pointed to by the arrow mark 44 represents the separation distance L.

For example, if the arrow mark 44 points to “0” as depicted in FIG. 12(a), the separation distance L is 0. On the other hand, if the arrow mark 44 points to “2” as depicted in FIG. 12(c), the separation distance L is 2 cm.

With the present fixing device 1, effects similar to those achieved by the aforementioned first embodiment are achieved.

Further, with the fixing device 1, the position of the urethral-insertion portion 31 with respect to the bladder can be fixed by the balloon 91 thereof.

Further, the separation distance L can be adjusted in response to a patient.

Further, in a state in which the fixing device 1 is mounted on a patient, the separation distance L can be grasped from the graduations 23 and the arrow mark 44.

### Third Embodiment

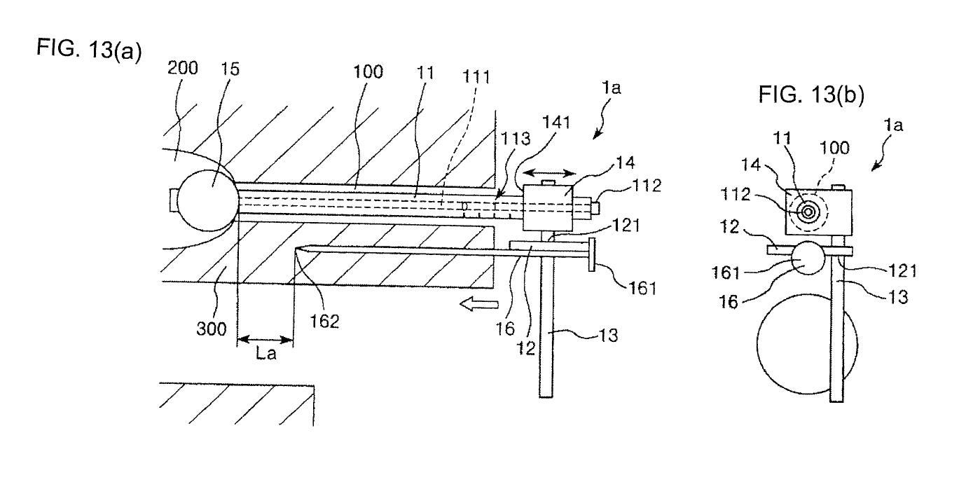

The drawing figures constituting FIG. 13 depict a third embodiment of the fixing device representing another example of the disclosed fixing device, wherein FIG. 13(a) is a side elevational view of the fixing device and FIG. 13(b) is a rear elevational view of the fixing device.

In the following description, the left side in FIG. 13(a) is referred to as the “distal end,” the right side as the “proximal end,” the upper side as the “upper end,” and the lower side as the “lower end.”

The following description of the third embodiment focuses principally on the differences relative to the first embodiment described above, and features of the third embodiment that are the same as those described above in the first embodiment are identified by common reference numerals and a detailed description of such features is not repeated.

As depicted in FIG. 13, a fixing device 1a of the third embodiment has a urethral-insertion portion 11 which is inserted into the urethra 100, a guide portion (guiding portion) 12, and an interlock portion 13 which interlocks the urethral-insertion portion 11 and the guide portion 12 with each other. The guide portion 12 is thus interlocked with or fixed to the urethral-insertion portion 11.

The urethral-insertion portion 11 and the interlock portion 13 individually have a form of a bar (bar-shaped), and a supporting portion 14 is disposed at an end portion of the urethral-insertion portion 11 at the right side in FIG. 13(a) for movement in a longitudinal direction of the urethral-insertion portion 11. Further, an end portion of the interlock portion 13 at the upper side in FIG. 13(a) is fixed to the supporting portion 14 so as to be orthogonal to the urethral-insertion portion 11.

An expandable and contractible balloon 15 is provided as a restriction unit, which restricts the position of the urethral-insertion portion 11 in the longitudinal direction in the urethra, at the distal portion of the urethral-insertion portion 11. When the fixing device 1a is used, the balloon 15 is inserted into a bladder 200 of a patient, and the balloon 15 is caught in an expanded state by the bladder neck thereby to fix the position of the urethral-insertion portion 11 with respect to the bladder 200 and the urethra 100.

Further, a lumen 111 is formed in the urethral-insertion portion 11. The distal end of the lumen 111 opens into the balloon 15 and the proximal end of the balloon 15 opens to a side face of the proximal portion of the urethral-insertion portion 11. Further, a port 112 which communicates with the proximal end of the lumen 111 is formed at the proximal portion of the urethral-insertion portion 11.

A balloon expanding tool like, for example, a syringe, is connectable to the port 112, and working fluid supplied from the balloon expanding tool is fed into or working fluid is pulled out from the balloon 15 through the lumen 111 to carry out expansion or contraction of the balloon 15. As the working fluid for balloon expansion, liquid such as, for example, physiological salt solution, gas and so forth can be used.

The guide portion 12 has a form of a plate. That is, the guide portion 12 is plate-shaped

Further, an opening 121 is formed at a central portion of the guide portion 12 in FIG. 13(a), and the interlock portion 13 is inserted in (passes through) the opening 121. Consequently, the guide portion 12 can move in a direction orthogonal to the interlock portion 13 along the interlock portion 13, and consequently, the distance between the guide portion 12 and the urethral-insertion portion 11 can be adjusted.

Since the guide portion 12 and the urethral-insertion portion 11 extend parallel to each other, the guide portion 12 and the urethra 100 extend parallel to each other.

A needle 16 guided by this guide portion 12 has a disk-shaped head portion 161 at the proximal end of the needle 16. When the needle 16 is to be moved in a direction toward the distal end to punctuate a vaginal wall 300, the needle 16 is slidably moved along a side face of the guide portion 12. Consequently, the needle 16 can be prevented from punctuating the urethra 100. Further, the head portion 161 of the needle 16 is attached to the proximal end of the guide portion 12, and consequently, the needle 16 stops and the position of the needle 16 is restricted.

Further, on an outer peripheral face of the urethral-insertion portion 11, graduations 113 are provided for indicating a separation distance La in the axial direction of the needle 16 between a needle point 162 of the needle 16 when the head portion 161 of the needle 16 attaches to or contacts the proximal end of the guide portion 12 and the balloon 15. Further, the graduations 113 are configured such that a graduation 113 pointed to by a distal end 141 of the supporting portion 14 represents the separation distance La. For example, if the distal end 141 of the supporting portion 14 points to “2” as depicted in FIG. 13(a), the separation distance La is 2 cm.

With the present fixing device 1a, effects similar to those achieved by the aforementioned first embodiment are achieved.

The present fixing device 1a is configured so that the position of the urethral-insertion portion 11 with respect to the bladder 200 can be fixed by the balloon 15.

Also, the separation distance La can be adjusted in response to the patient.

Further, the separation distance La can be grasped by the graduation 113 in a state in which the fixing device 1a is mounted on the patient.

Although the fixing device disclosed here has been described based on the embodiments depicted in the drawings, the present invention is not limited to this. The configuration of the components can be replaced by an arbitrary configuration having similar functions. Further, arbitrary components may be added.

The fixing device may be configured to include a combination of features of two or more configurations of the embodiments.

In the embodiments described above, a case in which the fixing device is applied to an apparatus which is used when an implant which can be buried for medical treatment of urinary incontinence in a woman is buried into the living body is described, the application of the fixing device is not limited to this.

The fixing device disclosed here can include a vaginal insertion portion of a longitudinal shape which is inserted into a vagina, and a urethral-insertion portion of a longitudinal shape which is interlocked with the vaginal insertion portion and inserted into a urethra, wherein the vaginal insertion portion has a guide portion which supports a medical tool of a longitudinal shape for movement along the longitudinal direction of the medical tool so as to guide the medical tool in the neighborhood of the urethra and in a direction (along a path of movement) in which the medical tool bypasses the urethra.

The fixing device disclosed here can also include a urethral-insertion portion of a longitudinal shape which is inserted into a urethra, a restriction unit provided on the urethral-insertion portion and restricting a position of the urethral-insertion portion in a longitudinal direction of the urethral-insertion portion in the urethra, and a guide portion interlocked with the urethral-insertion portion and supporting a medical tool of a longitudinal shape for movement along a longitudinal direction of the medical tool so as to guide the medical tool in the proximity of the urethra and in a direction in which the medical tool bypasses the urethra.

With the fixing device disclosed here, when an implant is buried, the burden on the patient is relatively light and the safety of the patient is rather high, and also the safety of the operator is high.

In particular, by inserting the vaginal insertion portion into the vagina and inserting the urethral-insertion portion into the urethra, the position of the urethra with respect to the vagina insertion portion is fixed to a particular position, and a medical tool of a longitudinal shape like a needle is guided by the guide portion provided on the vaginal insertion portion. Therefore, the needle can be prevented from puncturing the urethra or the bladder and can be prevented from puncturing a fingertip of the operator.

The detailed description above describes, by way of example, several embodiments of a fixing device. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A fixing device comprising:
a vaginal insertion portion configured to be inserted into a vagina, the vaginal insertion portion possessing an elongated shape; and
a urethral-insertion portion configured to be inserted into a urethra, the urethral-insertion portion possessing an elongated shape and being interlocked with the vaginal insertion portion; and
wherein the vaginal insertion portion includes a guide portion configured to support a medical tool which possesses a longitudinal shape for movement along a longitudinal direction of the medical tool so as to guide the medical tool adjacent a urethra and along a path of movement in which the medical tool bypasses the urethra.

2. The fixing device according to claim 1, wherein the guide portion is configured to guide a plurality of the medical tools along respective paths of movement that are different from each other.

3. The fixing device according to claim 1, wherein the guide portion includes a guide hole extending completely through the vaginal insertion portion.

4. The fixing device according to claim 3, wherein an extension line of the guide hole does not cross the urethral-insertion portion.

5. The fixing device according to claim 1, wherein the guide portion includes two spaced apart guide holes each extending completely through the vaginal insertion portion, and the two guide holes being arranged so that respective extension lines of the two guide holes do not individually cross the urethral-insertion portion.

6. The fixing device according to claim 5, wherein the extension lines of the two guide holes cross each other at a location between the vaginal insertion portion and the urethral-insertion portion.

7. The fixing device according to claim 1, wherein the vaginal insertion portion and the urethral-insertion portion are separated by a separation distance, and further comprising adjustment means for adjusting the separation distance between the vaginal insertion portion and the urethral-insertion portion.

8. The fixing device according to claim 1, wherein the urethral-insertion portion is movably mounted on the vaginal insertion portion for movement relative to the vaginal insertion portion along a longitudinal extent of the vaginal insertion portion.

9. The fixing device according to claim 1, wherein the vaginal insertion portion is plate-shaped.

10. The fixing device according to claim 1, wherein the vaginal insertion portion is curved along its longitudinal extent.

11. The fixing device according to claim 10, wherein the urethral-insertion portion is curved along its longitudinal extent, the urethral-insertion portion and the vaginal insertion portion being curved in a common direction.

12. A fixing device comprising:
a urethral-insertion portion configured to be inserted into a urethra, the urethral-insertion portion possessing an elongated shape;
a restriction unit on the urethral-insertion portion that restricts a position of the urethral-insertion portion, in a longitudinal direction of the urethral-insertion portion, in the urethra; and
a guide portion interlocked with the urethral-insertion portion to movably support a medical tool possessing an elongated shape for movement along a longitudinal extent of the medical tool to guide the medical tool adjacent a urethra and along a path of movement in which the medical tool bypasses the urethra.

13. The fixing device according to claim 12, wherein the restriction unit is disposed at a distal end side of the urethral-insertion portion.

14. The fixing device according to claim 12, wherein the restriction unit is an expandable and contractible balloon.

15. The fixing device according to claim 12, further comprising adjustment means for adjusting a separation distance, in a longitudinal direction of the urethral-insertion, between the restriction unit and a position of the urethral-insertion portion.

16. The fixing device according to claim 12, further comprising graduations identifying a separation distance, in a longitudinal direction of the urethral-insertion portion, between the restriction unit and a position of the urethral-insertion portion.

17. The fixing device according to claim 16, wherein the graduations are located on the urethral-insertion portion.

18. A method of inserting an elongated medical tool into a living body that includes a urethra comprising:
inserting an elongated urethral insertion portion into the urethra of the living body;
puncturing the living body with the medical tool by moving the elongated medical tool in a direction along a longitudinal extent of the medical tool; and
guiding the elongated medical tool along a guide portion while the medical tool is moved in the direction along the longitudinal extent of the medical tool so that the medical tool is moved adjacent the urethra in the living body and along a path of movement in which the medical tool bypasses the urethra.

19. The method according to claim 18, further comprising inserting an elongated vaginal insertion portion into a vagina of the living body, and the guiding of the medical tool along the guide portion including guiding the medical tool along a through hole in the elongated vaginal insertion portion.

20. The method according to claim 18, including an interlock portion movable along the elongated urethral insertion portion, the guide portion being fixed to the interlock portion and being parallel to the elongated urethral insertion portion to guide the medical tool in a direction parallel to the elongated urethral insertion portion.
